(19) United States
(12) Patent Application Publication
(10) Pub. No.: US 2026/0165580 A1
(43) Pub. Date: Jun. 18, 2026

(54) AUTOMATIC CATARACT GRADING BASED ON THREE-DIMENSIONAL OPTICAL COHERENCE TOMOGRAPHY

(71) Applicant: Alcon Inc., Fribourg (CH)

(72) Inventors: Varun Magesh Iyer, Pleasanton, CA (US); Chad P. Byers, Mission Viejo, CA (US); Mark Andrew Zielke, Lake Forest, CA (US); Lance Noller, Yorba Linda, CA (US); Zhaokai Meng, Aliso Viejo, CA (US); George Hunter Pettit, Fort Worth, TX (US)

(21) Appl. No.: 19/411,535

(22) Filed: Dec. 8, 2025

Related U.S. Application Data
(60) Provisional application No. 63/735,017, filed on Dec. 17, 2024.

(51) Int. Cl. A61B 3/117; A61B 3/10; A61F 9/007; A61F 9/008; G06T 7/00

(52) U.S. Cl. CPC A61B 3/1176; A61B 3/102; A61F 9/00745; A61F 9/008; G06T 7/0014; A61F 2009/00887; G06T 2207/10101; G06T 2207/30041

(57) ABSTRACT

A system and method of automatic cataract grading uses an optical coherence tomography (“OCT”) device to generate three-dimensional OCT data of a lens. The system includes a controller having at least one processor and at least one non-transitory, tangible memory on which instructions are recorded. The controller is configured to divide the lens into a plurality of regions, and define segmentation lines for the plurality of regions based in part on predetermined reference values. The controller is configured to adjust the segmentation lines to fit respective areas adjacent to the plurality of regions, the respective areas having a relatively high gradient value. A respective mean opacity for the plurality of regions is determined based on the volumetric OCT data. The controller is configured to generate a cataract distribution trace of the lens based on the respective mean opacity in the plurality of regions.

### INTRODUCTION

The disclosure relates generally to automatic cataract grading based on three-dimensional optical coherence tomography. Optical coherence tomography (“OCT”) is a noninvasive imaging technology using low-coherence interferometry to generate high-resolution images of ocular structure. OCT imaging functions partly by measuring the echo time delay and magnitude of backscattered light. Images generated by OCT are useful for many purposes, such as identification and assessment of ocular diseases. OCT images are frequently taken prior to cataract surgery, where an intraocular lens is implanted into a patient's eye. Cataract grading for surgical decision-making and planning is generally based on a surgeon's analysis of data, such as for example, a slit-lamp or photographic examination of a patient. These approaches depend on highly trained and consistent practitioners, and are subjective.

### SUMMARY

Disclosed herein is a system and method of automatic cataract grading a using an optical coherence tomography (“OCT”) device. The system includes a controller having at least one processor and at least one non-transitory, tangible memory on which instructions are recorded. The OCT device generate three-dimensional OCT data of a lens. Execution of the instructions by the processor causes the controller to divide the lens into a plurality of regions, and define segmentation lines for the plurality of regions based in part on predetermined reference values. The controller is configured to adjust the segmentation lines to fit respective areas adjacent to the plurality of regions, where the respective areas have a relatively high gradient value. A respective mean opacity for the plurality of regions is determined based on the three-dimensional OCT data. The controller is configured to generate a cataract distribution trace of the lens based on the respective mean opacity in the plurality of regions.

The controller may be adapted to obtain a respective standard deviation for the plurality of regions. The plurality of regions may include a nuclear region, a posterior cortical region, an anterior cortical region, and a posterior subcapsular region. In some embodiments, the OCT device includes a swept-source OCT. The plurality of regions may include a first series of parallel planes extending along an axial direction. The plurality of regions may include a second series of parallel planes extending along a direction perpendicular to an axial direction.

In some embodiments, the controller is configured to define an axis of interest in the lens such that relatively high value points in the cataract distribution trace are approximately symmetrical; determine a respective mean intensity in a sweep of points around the axis of interest; and augment the cataract distribution trace of the lens based on the mean intensity along the axis of interest.

The controller may be configured to: calculate variance statistics for the respective mean intensity; and compare respective shapes of the respective mean intensity around the axis of interest with predefined patterns from a reference dataset. The system may include a laser unit adapted to selectively generate a laser treatment beam directed towards the lens, the laser treatment beam being adjusted based in part on the cataract distribution trace. The laser treatment beam includes a plurality of ultra-short laser pulses, the plurality of ultra-short laser pulses defining a respective time duration of between about a femtosecond and about 50 picoseconds. The system may include a phacoemulsification unit adapted to selectively generate an ultrasonic treatment beam directed towards the lens, the ultrasonic treatment beam being adjusted based in part on the cataract distribution trace.

Disclosed herein is a method for automatic cataract grading using an optical coherence tomography (OCT) device in a system having a controller with at least one processor and at least one non-transitory, tangible memory. The method includes dividing a lens into a plurality of regions, the OCT device being adapted to generate three-dimensional OCT data of the lens. The method includes defining respective segmentation lines for the plurality of regions based in part on predetermined reference values; and adjusting the respective segmentation lines to fit respective areas adjacent to the plurality of regions, the respective areas having a relatively high gradient value. The method includes determining a respective mean opacity for the plurality of regions based on the three-dimensional OCT data; and generating a cataract distribution trace of the lens based on the respective mean opacity in the plurality of regions.

The method may include obtaining a respective standard deviation for the respective mean opacity, via the controller. The plurality of regions may include a nuclear region, a posterior cortical region, an anterior cortical region, and a posterior subcapsular region. The plurality of regions may include a first series of parallel planes extending along an axial direction. The plurality of regions may include a second series of parallel planes extending along a direction perpendicular to an axial direction.

In some embodiments, the method includes defining an axis of interest in the lens such that relatively high value points in the cataract distribution trace are approximately symmetrical; determining a respective mean intensity in a radial sweep of points around the axis of interest; calculating variance statistics for the respective mean intensity; comparing respective shapes of the respective mean intensity around the axis of interest with predefined patterns from a reference dataset; and augmenting the cataract distribution trace of the lens based on the mean intensity along the axis of interest.

The method may include adjusting parameters of a laser treatment beam directed towards the lens based in part on the cataract distribution trace, the laser treatment beam being selectively generated by a laser unit. The method may include adjusting parameters of an ultrasonic treatment beam directed towards the lens based in part on the cataract distribution trace, the ultrasonic treatment beam being selectively generated by a phacoemulsification unit.

The above features and advantages and other features and advantages of the present disclosure are readily apparent from the following detailed description of the best modes for carrying out the disclosure when taken in connection with the accompanying drawings.

### BRIEF DESCRIPTION OF THE DRAWINGS

FIG. 1 is a schematic illustration of a system for automatic cataract grading, the system having a controller and an OCT device;

FIG. 2 is a schematic diagram of a lens in an eye, illustrating segmentation into a plurality of regions;

FIG. 3 is a schematic flowchart for a method executable by the controller of FIG. 1;

FIGS. 4A-C are schematic diagrams illustrating example scanning regions for the OCT device of FIG. 1;

FIG. 5 is a schematic diagram of a first set of planes through the lens, each perpendicular to a first reference plane;

FIG. 6 is a schematic diagram of a second set of planes through the lens, each perpendicular to a second reference plane;

FIG. 7 is an example graph showing signal amplitude on the vertical axis and dimension or depth on the horizontal axis;

FIG. 8 is a schematic flowchart for another method executable by the controller of FIG. 1; and

FIG. 9 is a schematic diagram illustrating various treatment options for the eye of FIG. 2.

Representative embodiments of this disclosure are shown by way of non-limiting example in the drawings and are described in additional detail below. It should be understood, however, that the novel aspects of this disclosure are not limited to the particular forms illustrated in the above-enumerated drawings. Rather, the disclosure is to cover modifications, equivalents, combinations, sub-combinations, permutations, groupings, and alternatives falling within the scope of this disclosure as encompassed, for instance, by the appended claims.

### DETAILED DESCRIPTION

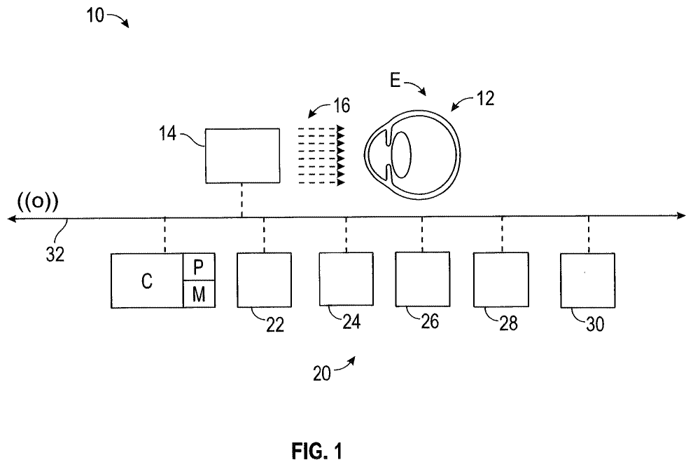

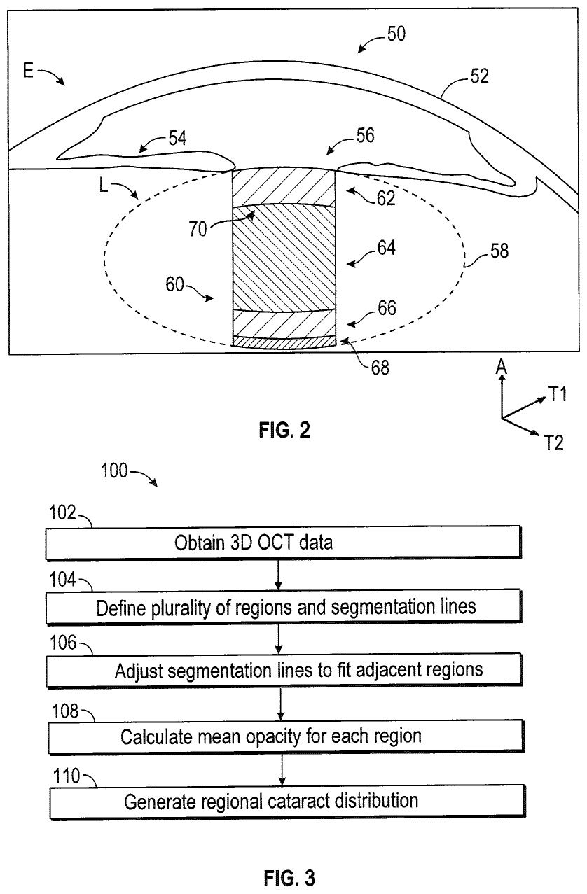

Referring to the drawings, wherein like reference numbers refer to like components, FIG. 1 schematically illustrates a system 10 that employs optical coherence tomography (“OCT” hereinafter) data of a target site 12, captured via an OCT device 14. The OCT device 14 may employ an array of laser beams 16 for illuminating the eye E to generate three-dimensional OCT data. The target site 12 here is an eye E, which is shown in greater detail in FIG. 2.

Cataract grading for surgical decision-making and planning is generally based on analysis of patient data by a surgeon. The data may include a slit-lamp or photographic examination of the patient's eye. These approaches depend on highly trained and consistent practitioners, and are subjective. As described below, the system 10 provides an objective, computed metric for automatic cataract grading that reduces dependence on trained practitioners. The system 10 implements a segmentation method to differentiate between different types of cataracts, such as between nuclear, cortical, and posterior more subcapsular cataracts.

Referring to FIG. 1, the system 10 includes a controller C having at least one processor P and at least one memory M (or non-transitory, tangible computer readable storage medium) on which instructions are recorded for executing method 100 which is shown in and described below with respect to FIG. 3. The method 100 enables comparison and correlation of different kinds of measurements to increase the overall accuracy and efficacy of produced metric.

Referring to FIG. 1, the controller C may be specifically programmed to selectively execute a plurality of learning modules 20, such as a first neural network 22, a second neural network 24, and a third neural network 26. The plurality of learning modules 20 may be embedded in the controller C or may be stored elsewhere and accessible to the controller C. Referring to FIG. 1, the learning modules 20 may be trained by a training network with multiple training datasets 28. The system 10 may be configured to be “adaptive” and may be updated periodically after the collection of additional data for the training datasets.

Referring now to FIG. 3, a flow chart of method 100 executable by the controller C of FIG. 1 is shown. Method 100 need not be applied in the specific order recited herein and some blocks may be omitted. The memory M can store controller-executable instruction sets, and the processor P can execute the controller-executable instruction sets stored in the memory M.

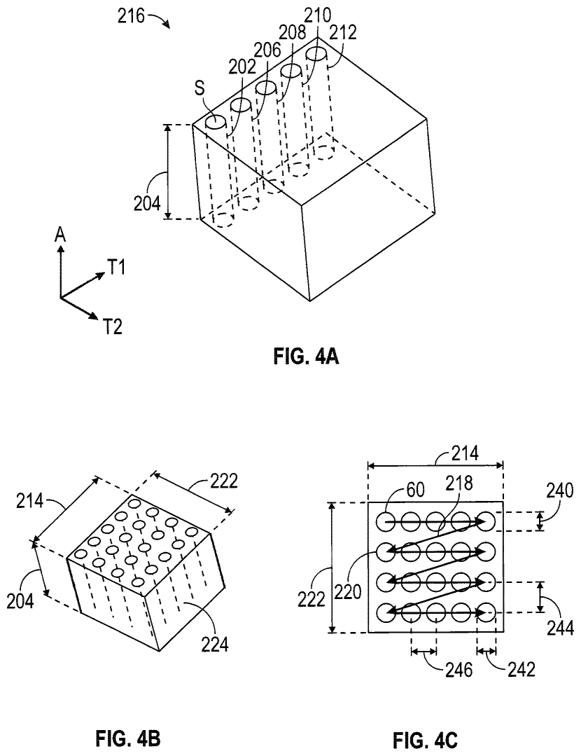

Per block 102 of FIG. 3, the controller C is configured to obtain the subject data from the OCT device 14. Referring now to FIGS. 4A-C, example scanning regions for the OCT device 14 are shown. FIGS. 4A and 4B are schematic fragmentary perspective views, while FIG. 4C is a schematic fragmentary top view of an example scanning pattern. Referring to FIG. 4A, a single scan directed at a spot S (in the target site 12) results in a depth scan 202 of the structure of the physical sample into which the beam 16 is directed, along the incident direction. Referring to FIG. 4A, the depth scan 202 may be referred to as an “A-scan” and is configured to scan to a detected depth 204 along an axial direction A. The axial direction A which is the travel direction of the light source (not shown) in the OCT device 14.

The OCT beam may be moved in a continual manner about the target site 12 using a steering unit (not shown) in the OCT device 14, thereby enabling a second depth scan 206, a third depth scan 208, a fourth depth scan 210 and a fifth depth scan 212 along a first transverse scan range 214, for example. Such a line of A-scans may be referred to as a B-scan or row scan 216. The sampling resolution of the system 10 is a function of the resolution in the axial direction A (the direction of the A-scan), the diameter of a single A-scan and the separation of adjacent A-scans in each of the two remaining directions, the first transverse direction T1 and the second transverse direction T2.

Referring to FIG. 4C, by steering the optical path appropriately along the first transverse scan range 214, then performing a “step-and-repeat” path steer along the raster pattern 218 to repeat the cycle at a starting point 220 and subsequent lines, a grid of depth scans may be traced out along the target site 12, along the first transverse scan range 214 and a second transverse scan range 222. Referring to FIGS. 4A and 4B, this results in a three-dimensional sampled volume having boundaries 224, which may have the shape of a cuboid.

The three-dimensional OCT data may include multiple A-scans per B-scan and multiple B-scans per three-dimensional volume. Once a three-dimensional volume has been acquired, an OCT enface image may be created by integrating intensity information along the axial direction, such that one summed A-scan represents a single pixel in the OCT image. Referring to FIG. 4C, the diameter of the spot scan S may be represented by a first set of dimensions 240, 242 and are related to factors such as the structure of the OCT source 40 and the optical path encountered by the laser beam 16. Referring to FIG. 4C, the separation of adjacent ones of the A-scans or depth scans 202 may be represented by a second set of dimensions 244, 246. The OCT device 14 may include a swept-source OCT. It is to be understood that the OCT device 14 may take many different forms and include multiple and/or alternate components.

Referring to FIG. 4A, the detected depth 204 or penetration depth for a depth scan 202 is dependent on many factors, including the spectrum of the OCT light source at the starting point 220, the optical characteristics of the starting point 220 over the spectrum and the spectral resolution of the detector system in the OCT device 14. Reflection points may appear as “bright” pixels in the line-scan camera data. For example, if the possible pixel values are in the range 0-255, non-reflection points might have a value of 25 or less, while bright reflection points might have a value of 125 or greater.

Also per block 102, the controller C is configured to divide the lens L into a plurality of regions 60, shown in FIG. 2. Referring to FIG. 2, an OCT image 50 of an eye E is shown with the sclera 52, iris 54, and pupil 56. OCT imaging does not capture the peripheral portion 58 of the lens L that is behind the iris 54 as the illuminating lasers used in OCT imaging cannot penetrate across the iris 54. However, OCT imaging techniques provide high resolution and a non-contact scanning method that consistently captures the entire depth of the lens L, regardless of cataract severity, with high resolution. Referring to FIG. 2, the plurality of regions 60 include an anterior cortical region 62, a nuclear region 64, a posterior cortical region 66, and a posterior subcapsular region 68.

Per block 104 of FIG. 3, the controller C is configured to define respective segmentation lines 70 for the plurality of regions 60 based in part on predetermined reference values, which may be accomplished using a machine learning module, such as the first neural network 22 in FIG. 1. Approximate segmentation lines for the plurality of regions 60 may be drawn using known average values for human lenses (through the training datasets 28). In one example, the first neural network 22 of FIG. 1 leverages convolutional neural network (CNN)-based deep learning techniques.

Per block 106 of FIG. 3, the method 100 includes adjusting the respective segmentation lines 70 to fit adjacent areas of the plurality of regions 60 that have a relatively high gradient value. In other words, the respective segmentation lines 70 are adjusted to separate adjacent areas with high gradient values. This may be accomplished using a machine learning module, such as a second neural network 24.

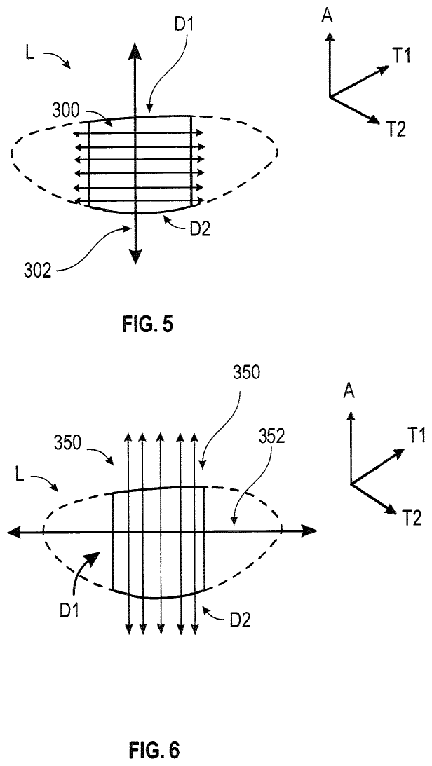

Referring to FIG. 5, in one embodiment, the plurality of regions 60 includes a first series of parallel planes 300, each perpendicular to a first reference plane 302 extending along the axial direction A. The first series of parallel planes 300 extends between a first end plane D1 and a second end plane D2, shown in FIG. 5. In one example, the first and second end planes D1, D2, may respectively correspond to the anterior surface and the posterior surface of the lens L.

Referring to FIG. 6, in another embodiment, the plurality of regions 60 includes a second series of parallel planes 350, each perpendicular to a second reference plane 352 in a direction that is perpendicular to the axial direction A. The second series of parallel planes 350 extends between a first end plane D1 and a second end plane D2, shown in FIG. 6. The thickness of the parallel planes 300, 350 may be varied based on the application at hand and may be selected to cover the span of the lens L between the first end plane D1 and the second end plane D2. In other words, if there are fewer parallel planes 300, 350, their respective thickness is increased to cover the span of the lens L.

Per block 108 of FIG. 3, the method 100 includes determine a respective mean opacity for the plurality of regions 60 based on the three-dimensional OCT data. The pixel values from the three-dimensional OCT data may be filtered to reduce noise or undergo other processing, e.g., smoothing. The controller C may be adapted to obtain a respective standard deviation value for the mean opacity. Other basic statistics may be computed on a per-region basis.

The mean opacity and respective standard deviation value may be used to indicate both cataract presence and the type/kind of cataract. For example, a clustered set of high opacity (with low standard deviation) may indicate a different type of cataract than a spread-out distribution (with high standard deviation) of high opacity. In addition, there is some correspondence between the region of the lenses which has the greatest opacity values and the kind of cataract the lens has been diagnosed with.

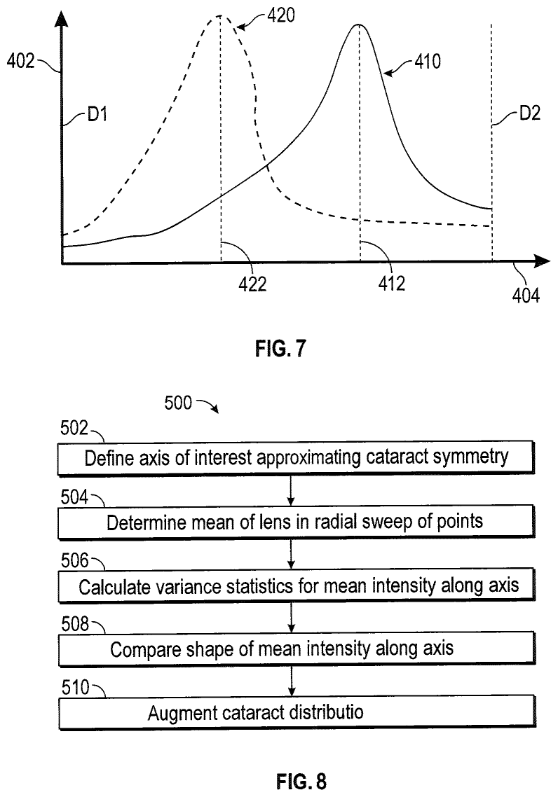

Per block 110 of FIG. 3, the controller C is configured to generate a cataract distribution trace of the lens L based on the respective mean opacity in the plurality of regions 60. Two example cataract distribution traces are shown in FIG. 7. FIG. 7 shows mean opacity values on the vertical axis 402 and dimension or distance on the horizontal axis 404. While the cataract distribution traces are shown in one dimension in FIG. 7, they may be expanded to generate cataract distribution traces along three dimensions. Each point on the cataract distribution trace represents the mean opacity determined for each plane in the series of parallel planes 300, 350 shown in FIGS. 5-6.

Referring to FIG. 7, the first cataract distribution trace 410 represents mean opacity between the first end plane D1 and the second end plane D2, with a higher opacity (indicating a cataract centered around axis 412) closer to the second end plane D2. The second cataract distribution trace 420 represents mean opacity between the first end plane D1 and the second end plane D2, with a higher opacity (indicating a cataract centered around axis 422) closer to the first end plane D1.

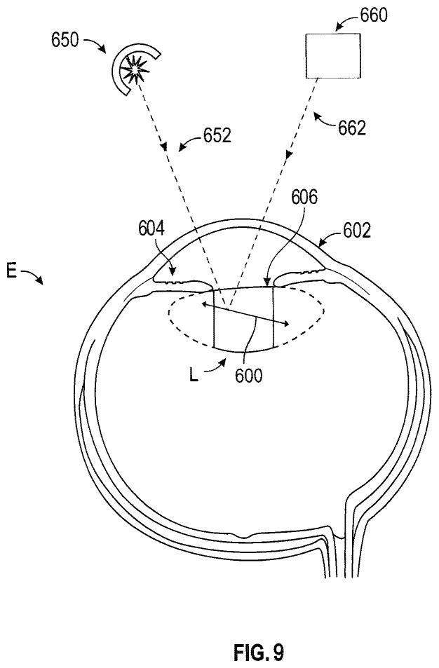

Referring now to FIG. 8, a flow chart of method 500 executable by the controller C of FIG. 1 is shown. Method 500 need not be applied in the specific order recited herein and some blocks may be omitted. Per block 502 of FIG. 8, the controller C is configured to obtain the subject data, via the OCT device 14, and define an axis of interest in the lens such that relatively high value points in the cataract distribution trace are approximately symmetrical. An example axis of interest 600 is shown in FIG. 9, in an eye E having a lens L, sclera 602, iris 604, and pupil 606. In other words, the axis of interest 600 is an approximate axis of symmetry through the cataract in the lens L.

Per block 504 of FIG. 8, the method 500 includes determining a respective mean intensity in a sweep of points e.g., radial sweep of points, sufficiently close or around the axis of interest 600. The radius of the sweep may be varied based on the application at hand and may be chosen to be a percentage of the overall width of the lens L. Per block 506 of FIG. 8, the controller C is configured to calculate variance statistics (e.g., standard deviation) for the respective mean intensity along the axis of interest 600.

Per block 508 of FIG. 8, the method 500 includes comparing respective shapes of the respective mean intensity around the axis of interest 600 with predefined patterns from a reference dataset. In other words, the shape of the mean intensity along the axis of interest 600 is compared with known patterns from reference healthy images and reference cataractous images, with known and specific types of cataracts. This may be accomplished using the third neural network 26, shown in FIG. 1. The mean intensity may be used as an indicator for the likelihood of different types or kinds of cataracts.

Per block 510 of FIG. 8, the controller C is configured to and augment the cataract distribution trace of the lens L based on the distribution of the mean intensity along the axis of interest 600.

Referring now to FIG. 9, a schematic diagram illustrating various treatment options for the eye E are shown. Referring to FIG. 9, a laser unit 650 is adapted to selectively generate a laser treatment beam 652 directed towards the lens L. The laser treatment beam 652 may include a plurality of ultra-short laser pulses, with the plurality of ultra-short laser pulses defining a respective time duration of between about a femtosecond and about 50 picoseconds. The direction, intensity, and other parameters of the laser treatment beam 652 may be adjusted based in part on the cataract distribution trace.

Referring to FIG. 9, a phacoemulsification unit 660 may be adapted to selectively generate an ultrasonic treatment beam 662 directed towards the lens L. The phacoemulsification unit 660 employs ultrasound energy to emulsify the targeted area. The direction, intensity, and other parameters of the ultrasonic treatment beam 662 may be adjusted based in part on the cataract distribution trace. The phacoemulsification unit 660 may include a handpiece, foot pedal, irrigation, and aspiration systems. Hard nuclear cataract detection and phacoemulsification parameter recommendations may aid in planning for surgical procedures. Phacoemulsification parameters may be tuned to minimize the total phacoenergy expended in the eye E while effectively aspirating the cataract.

In summary, the system 10 illustrates a robust way to obtain a variety of metrics intended to communicate the severity and nature of the cataract to the practitioner. These metrics may be used to (1) communicate the situation to the patient and to other practitioners, (2) make decisions regarding surgery or other interventions, and (3) plan the surgical procedure.

The controller C may be configured to receive and transmit data through a user interface 30, shown in FIG. 1. The user interface 30 may be installed on a smartphone, laptop, tablet, desktop or other electronic device and may include a touch screen interface or I/O device such as a keyboard or mouse. The user interface 30 may be a mobile application. The circuitry and components of a mobile application (“apps”) available to those skilled in the art may be employed. The user interface 30 may include an integrated processor and integrated memory.

The various components of the system 10 of FIG. 1 may communicate via a wireless network 32, which may be a short-range network or a long-range network. The wireless network 32 may be a bus implemented in various ways, such as for example, a serial communication bus in the form of a local area network. The local area network may include, but is not limited to, a Controller Area Network (CAN), a Controller Area Network with Flexible Data Rate (CAN-FD), Ethernet, blue tooth, WIFI and other forms of data connection. The wireless network 32 may be a Wireless Local Area Network (LAN) which links multiple devices using a wireless distribution method, a Wireless Metropolitan Area Networks (MAN) which connects several wireless LANs or a Wireless Wide Area Network (WAN) which covers large areas such as neighboring towns and cities. Other types of connections may be employed.

The controller C of FIG. 1 includes a computer-readable medium (also referred to as a processor-readable medium), including a non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random-access memory (DRAM), which may constitute a main memory. Such instructions may be transmitted by one or more transmission media, including coaxial cables, copper wire and fiber optics, including the wires that comprise a system bus coupled to a processor of a computer. Some forms of computer-readable media include, for example, a floppy disk, a flexible disk, hard disk, magnetic tape, other magnetic medium, a CD-ROM, DVD, other optical medium, a physical medium, a RAM, a PROM, an EPROM, a FLASH-EEPROM, other memory chip or cartridge, or other medium from which a computer can read.

Look-up tables, databases, data repositories or other data stores described herein may include various kinds of mechanisms for storing, accessing, and retrieving various kinds of data, including a hierarchical database, a set of files in a file storage system, an application database in a proprietary format, a relational database energy management system (RDBMS), etc. Each such data store may be included within a computing device employing a computer operating system such as one of those mentioned above and may be accessed via a network in one or more of a variety of manners. A file system may be accessible from a computer operating system and may include files stored in various formats. An RDBMS may employ the Structured Query Language (SQL) in addition to a language for creating, storing, editing, and executing stored procedures, such as the PL/SQL language mentioned above.

The flowchart shown in the FIGS. illustrates an architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by specific purpose hardware-based systems that perform the specified functions or acts, or combinations of specific purpose hardware and computer instructions. These computer program instructions may also be stored in a computer-readable medium that can direct a controller or other programmable data processing apparatus to function in a particular manner, such that the instructions stored in the computer-readable medium produce an article of manufacture including instructions to implement the function/act specified in the flowchart and/or block diagram blocks.

The numerical values of orders (e.g., of quantities or conditions) in this specification, including the appended claims, are to be understood as being modified in each respective instance by the term “about” whether or not “about” actually appears before the numerical value. “About” indicates that the stated numerical value allows some slight imprecision (with some approach to exactness in the value; about or reasonably close to the value; nearly). If the imprecision provided by “about” is not otherwise understood in the art with this ordinary meaning, then “about” as used herein indicates at least variations that may arise from ordinary methods of measuring and using such orders. In addition, disclosure of ranges includes disclosure of each value and further divided ranges within the entire range. Each value within a range and the endpoints of a range are hereby disclosed as separate embodiments.

The detailed description and the drawings or FIGS. are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. A system of automatic cataract grading using an optical coherence tomography (“OCT”) device, the system comprising:
a controller having at least one processor and at least one non-transitory, tangible memory on which instructions are recorded, the OCT device being adapted to generate three-dimensional OCT data of a lens;
wherein execution of the instructions by the processor causes the controller to:
divide the lens into a plurality of regions, and define respective segmentation lines for the plurality of regions based in part on predetermined reference values;
adjust the respective segmentation lines to fit respective areas adjacent to the plurality of regions, the respective areas having a relatively high gradient value;
determine a respective mean opacity for the plurality of regions based on the three-dimensional OCT data; and
generate a cataract distribution trace of the lens based on the respective mean opacity in the plurality of regions.

2. The system of claim 1, wherein the controller is adapted to obtain a respective standard deviation for the plurality of regions.

3. The system of claim 1, wherein the plurality of regions include a nuclear region, a posterior cortical region, an anterior cortical region, and a posterior subcapsular region.

4. The system of claim 1, wherein the OCT device includes a swept-source OCT.

5. The system of claim 1, wherein the plurality of regions includes a first series of parallel planes extending along an axial direction.

6. The system of claim 1, wherein the plurality of regions includes a second series of parallel planes extending along a direction perpendicular to an axial direction.

7. The system of claim 1, wherein the controller is configured to:
define an axis of interest in the lens such that relatively high value points in the cataract distribution trace are approximately symmetrical;
determine a respective mean intensity in a sweep of points around the axis of interest; and
augment the cataract distribution trace of the lens based on the mean intensity along the axis of interest.

8. The system of claim 7, wherein the controller is configured to:
calculate variance statistics for the respective mean intensity; and
compare respective shapes of the respective mean intensity around the axis of interest with predefined patterns from a reference dataset.

9. The system of claim 1, further comprising:
a laser unit adapted to selectively generate a laser treatment beam directed towards the lens, the laser treatment beam being adjusted based in part on the cataract distribution trace.

10. The system of claim 9, wherein the laser treatment beam includes a plurality of ultra-short laser pulses, the plurality of ultra-short laser pulses defining a respective time duration of between about a femtosecond and about 50 picoseconds.

11. The system of claim 1, further comprising:
a phacoemulsification unit adapted to selectively generate an ultrasonic treatment beam directed towards the lens, the ultrasonic treatment beam being adjusted based in part on the cataract distribution trace.

12. A method for automatic cataract grading using an optical coherence tomography (OCT) device in a system having a controller with at least one processor and at least one non-transitory, tangible memory, the method comprising:
dividing a lens into a plurality of regions, the OCT device being adapted to generate three-dimensional OCT data of the lens;
defining respective segmentation lines for the plurality of regions based in part on predetermined reference values;
adjusting the respective segmentation lines to fit respective areas adjacent to the plurality of regions, the respective areas having a relatively high gradient value;
determining a respective mean opacity for the plurality of regions based on the three-dimensional OCT data; and
generating a cataract distribution trace of the lens based on the respective mean opacity in the plurality of regions.

13. The method of claim 12, further comprising:
obtaining a respective standard deviation for the respective mean opacity, via the controller.

14. The method of claim 12, further comprising:
including a nuclear region, a posterior cortical region, an anterior cortical region, and a posterior subcapsular region in the plurality of regions.

15. The method of claim 12, further comprising:
including a first series of parallel planes in the plurality of regions, the first series extending along an axial direction.

16. The method of claim 12, further comprising:
including a second series of parallel planes in the plurality of regions, the second series extending along a direction perpendicular to an axial direction.

17. The method of claim 12, further comprising:
defining an axis of interest in the lens such that relatively high value points in the cataract distribution trace are approximately symmetrical;
determining a respective mean intensity in a radial sweep of points around the axis of interest;
calculating variance statistics for the respective mean intensity;
comparing respective shapes of the respective mean intensity around the axis of interest with predefined patterns from a reference dataset; and
augmenting the cataract distribution trace of the lens based on the mean intensity along the axis of interest.

18. The method of claim 17, further comprising:
adjusting parameters of a laser treatment beam directed towards the lens based in part on the cataract distribution trace, the laser treatment beam being selectively generated by a laser unit.

19. The method of claim 17, further comprising:
adjusting parameters of an ultrasonic treatment beam directed towards the lens based in part on the cataract distribution trace, the ultrasonic treatment beam being selectively generated by a phacoemulsification unit.

20. A system of automatic cataract grading using an optical coherence tomography (“OCT”) device, the system comprising:
a controller having at least one processor and at least one non-transitory, tangible memory on which instructions are recorded, the OCT device generating three-dimensional OCT data of a lens;
wherein execution of the instructions by the processor causes the controller to:
divide the lens into a plurality of regions, including a nuclear region, a posterior cortical region, an anterior cortical region, and a posterior subcapsular region;
define respective segmentation lines for the plurality of regions based in part on predetermined reference values;
adjust the respective segmentation lines to fit respective areas adjacent to the plurality of regions, the respective areas having a relatively high gradient value;
determine a respective mean opacity for the plurality of regions based on the three-dimensional OCT data;
generate a cataract distribution trace of the lens based on the respective mean opacity in the plurality of regions;
define an axis of interest in the lens such that relatively high value points in the cataract distribution trace are approximately symmetrical;
determine a respective mean intensity in a sweep of points around the axis of interest; and
augment the cataract distribution trace of the lens based on the respective mean intensity along the axis of interest.
